(19) United States
(12) Patent Application Publication
(10) Pub. No.: US 2024/0368514 A1
(43) Pub. Date: Nov. 7, 2024

(54) SBS-FORMAT SINGLE, OR DUAL, DEEP WELL STACKABLE CELL CULTURE DISPOSABLE WELL PLATE AND CLOSURE

(71) Applicant: Kuhner Shaker Inc., San Carlos, CA (US)

(72) Inventor: Robert David Laidlaw, Vernon (CA)

(21) Appl. No.: 18/653,693

(22) Filed: May 2, 2024

Related U.S. Application Data
(60) Provisional application No. 63/500,553, filed on May 5, 2023.

(51) Int. Cl. C12M 3/00; C12M 1/00

(52) U.S. Cl. CPC C12M 23/44; C12M 23/28; C12M 23/38; C12M 23/40; C12M 23/42

(57) ABSTRACT

The invention is a cell culture container having a top, bottom and sidewalls defining an interior region and at least one cultivation vessel disposed within the container, the cultivation vessel has a generally frustoconical shape. A cover member is provided for closing the top of the container and sealing the at least one cultivation vessel. The cover member and the bottom of the container are adapted to interfit so that multiple cell culture containers may be stacked on top of each other.

### CROSS-REFERENCE TO RELATED APPLICATIONS

This application claims the benefit of priority from U.S. Provisional Application Ser. No. 63/500,553 filed on May 5, 2023, which is incorporated herein by reference.

### FIELD OF THE INVENTION

The present invention is in the field of life-sciences and in particular, laboratory applications that cultivate microbial, plant, human and animal cells.

### BACKGROUND OF THE INVENTION

Shaker incubators are used in laboratories, research centers and production facilities for the in vitro cultivation of cells. The incubator shaker will receive a series of individual flasks (cultivation vessels) including rectangular well plates and cylindrical test tubes (tubes) containing a suspension of cells and growth media. The purpose of the cultivation vessel is to contain the cells and growth media and to allow gas exchange to support growth of the cells. The vessel must contain the cells and media and be hermetically sealed from the external environment, preventing unwanted contaminants from the incubator environment from entering the vessel. Cultivation vessels are placed into the shaker incubator onto a shaker tray, where they are secured during the cultivation process. Rectangular well plates may have a plurality of wells (vessels) commonly including 6, 24 or 96 vessel capacity; oftentimes larger well plate vessels are also placed inside the incubator onto a tray and secured for cultivation. The cultivation vessels are mechanically (orbitally) shaken while parameters including temperature, carbon dioxide and humidity within the incubator are controlled to promote cell growth.

Cell cultivation often requires cultivation vessels of varying size and shape. For example, rectangular well plates are most common for small volume (<0.50 ml) cultivations whereas conical bottom, cylindrical, vented cap tubes, known as tube spins, are desirable for mid-volume (5.0-35.0 ml) cell cultivation strategies and Erlenmeyer “shake” flasks are used for volumes greater than 20.0 milliliters. Round, cylindrical and non-rectangular cultivation vessels require an automation suite of highly specialized robotics to handle their unique size and shape, to manipulate them into and out of the incubator, and to cap and de-cap each of the vessels, etc. Very often these steps must be performed manually if an Erlenmeyer type flask is the cultivation vessel.

Well plates for the purpose of cultivating cells are of two primary heights and are referred to as ‘low well’ plates and ‘deep well’ plates. Low well plates have an approximate height of 14.4 mm. Deep well plates have an approximate height of 42 mm. Deep well plates, owing to the larger volume of the resulting wells (vessels) allow larger volumes of cultures to be deposited into each well of the well plate. Larger culture volumes are often needed for subsequent sample analysis after the cultivation period is complete. Established robotics and sampling systems are designed to work with well plate vessels of both the low well and deep well standardized heights allowing for easy adoption of vessels sharing these dimensions into automated workflows.

Disposable cultivation vessels (available for well plates, tubes and flasks) improve workflow speeds (no need for cleaning or washing vessels) and de-risk product cross contamination and (if pre-sterilized) eliminate sterility concerns. Disposable vessels for cell culture are often individually packaged and pre-sterilized to guarantee contaminating organisms are not present. Disposable vessels are often made of plastics such as Polycarbonate, Polystyrene, Polyethylene terephthalate Glycol and Polypropylene, although other polymers are also common in the industry.

A problem with automated systems is that they must be tailored to the type of cultivation vessel being handled. This means automation devices in other parts of the workflow are likely not compatible if vessels do not share the same shape. For example, cultivation vessels having a rounded or conical shape cannot be handled by gripper and manipulating systems designed for rectangular standardized format containers; namely, the SBS-format (Society for Biomolecular Screening) that are in use upstream from the shaker incubator. SBS-format is an internationally agreed upon housing for well plates that has a uniform size and shape and for which automation hardware has long existed. Since the rectangular dimensions of an SBS-format container are always the same, off the shelf robotics, landing platforms, and stacking systems may be used. In other words, compatibility with SBS-format eliminates the need for the specialized robotics necessary to handle unusual shapes and sizes of cultivation vessels, including larger (>35.0 ml) cultivation vessels with round shapes and round bottoms such as Erlenmeyer cultivation vessels. As is apparent, designing and creating specialized robotics to handle such vessels is undesirable as it would require a large expenditure of capital.

A need has existed in the art for a disposable well plate container that is adapted to hold relatively large round and conical cultivation vessels yet is capable of being handled by standard SBS compliant robotics, landing platforms, and stacking systems.

### BRIEF SUMMARY OF THE INVENTION

The present invention is a cell culture container having a top, bottom and sidewall that define an interior region, the top having an opening to the interior region, at least one cultivation vessel is in the container, the cultivation vessel has an open top, a bottom and a sidewall that define an interior region, each of the open top, bottom and sidewall has a diameter, the diameters of the bottom and open top are less than the diameter of the sidewall and the sidewall has a generally frustoconical shape. The container is stackable onto other containers and includes an optional cover fitted to the top of the container which hermetically seals the vessels and includes a vent that permits gas to exit the at least one cultivation vessel.

In another embodiment the invention is a culture container comprising a top, bottom and four sidewalls that define an interior region, the top having an opening to the interior region and the container has a rectangular shape, a pair of cultivation vessels are provided in the container and each of the pair of cultivation vessels has an open top, a bottom and a sidewall that define an interior region, each of the pair of cultivation vessels open tops, bottoms, and sidewalls have a diameter, the diameters of the bottoms and open tops are less than the diameters of the sidewalls, the sidewalls have a generally frustoconical shape, and each cultivation vessel is in contact with a separate pair of the four sidewalls.

In another embodiment the invention is a culture container comprising a top, a bottom and a sidewall that define an interior region, the top having an opening to the interior region. At least one cultivation vessel is disposed in the container, the at least one cultivation vessel having an open top, a bottom and a sidewall that define an interior region, the at least one cultivation vessel sidewall having an upper portion of a generally frustoconical shape and a lower portion of a generally frustospherical shape, the at least one cultivation vessel open top, bottom and sidewall has a diameter, the diameters of the bottom and open top are less than the diameter of the sidewall where the upper and lower portion meet.

In another embodiment, the invention incorporates a manual shake flask shape into an ANSI/SBS-format (American National Standards Institute/Society for Biomolecular Screening) automatable well plate. This allows conventional ‘shake flask’ experiments-that are typically accomplished by hand-to now be accomplished using automation. Users may also stack the conventional shake flask cultivations vertically to permit a greater number of cultivations per cultivation shaker incubator tray. The invention transforms manual handling of mid-scale, orbitally shaken, biological experiments into high experiment density, automatable, SBS format deep well containers.

### BRIEF DESCRIPTION OF THE SEVERAL VIEWS OF THE DRAWINGS

FIG. 1 is a perspective view of one embodiment of the present invention with two cultivation vessels and with portions of the container shown in phantom lines;

FIG. 2 is a side plan view of a cultivation vessel according to the present invention;

FIG. 3 is a perspective view of the present invention showing two FIG. 1 containers aligned in a stacked arrangement and with portions of the cultivations vessels and the container depicted in phantom lines;

FIG. 4 is an elevated perspective view of the container shown in FIG. 1 with the cultivation vessels removed;

FIG. 5 is an elevated perspective view of the invention shown in FIG. 1 in combination with a closure/cover member and with portions of the cultivation vessels and the container shown in phantom lines;

FIG. 6 is a perspective view showing the FIG. 5 container aligned in stacked arrangement with additional containers and with portions of the cultivations vessels and the containers shown in phantom lines;

FIG. 7 is an elevated perspective view of another embodiment of the present invention with a single cultivation vessel and with portions of the cultivation vessel and the container shown in phantom lines;

FIG. 8 is a perspective view of another embodiment of the present invention with a closure/cover member;

FIG. 9 is an exploded perspective view of the invention shown in FIG. 8;

FIG. 10 is a perspective view of the invention shown in FIG. 8 with the closure/cover member apart from the container;

FIG. 11 is a top perspective view of the container shown in FIG. 8;

FIG. 12 is a bottom perspective view of the container shown in FIG. 11;

FIG. 13 is a bottom perspective view of the closure/cover shown in FIGS. 8 and 10;

FIG. 14 is a top perspective view of the invention shown in FIG. 8 when in a stacked relation with additional containers;

FIG. 15 is a cross-sectional view of the invention taken along lines 15-15 in FIG. 14;

FIG. 16 is a top perspective view of another embodiment of the closure/cover according to the present invention;

FIG. 17 is a cross-sectional view of the invention taken along lines 17-17 in FIG. 4;

FIG. 18 is a top perspective view of another embodiment of the closure/cover member according to the present invention;

FIG. 19 is a bottom perspective of the closure/cover member shown in FIG. 18;

FIG. 20 is an exploded perspective view of the closure/cover member shown in FIG. 18;

FIG. 21 is an exploded view of the closure/cover member shown in FIG. 19;

FIG. 22 is a side elevational view of the closure/cover member shown in FIG. 18; and

FIG. 23 is cross-sectional view of the cover/closure shown in FIG. 18.

### DETAILED DESCRIPTION OF THE INVENTION

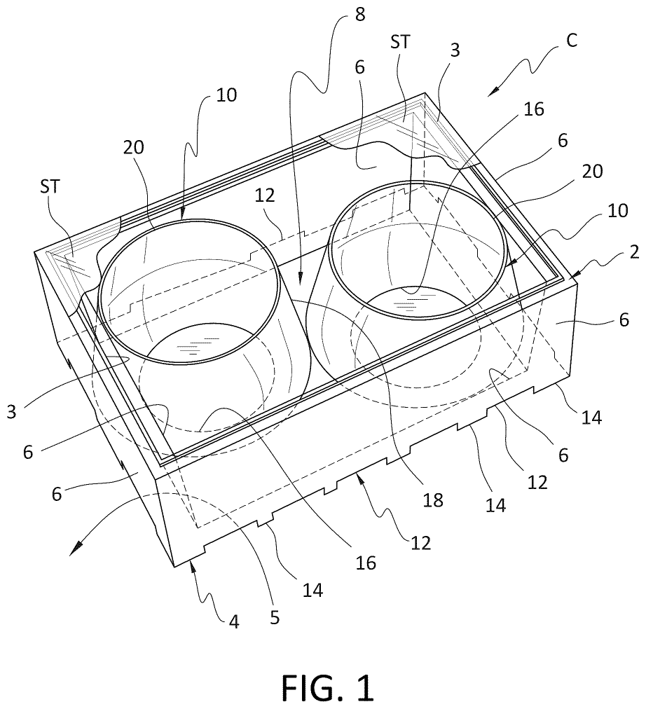

FIGS. 1 through 4 illustrate an embodiment of the well plate container C according to the present invention which is shown to have an open top 2, a bottom 4 and side walls 6 defining an interior container region 8. A pair of cultivation vessels 10 are disposed within the interior 8 of the container C. The top 2 of the container is provided with a raised edge 3 that extends upwardly from its surface.

Preferred construction materials of the well plate container include Polycarbonate (PC), Polystyrene (PS), Polyethylene terephthalate Glycol (PETG), Polypropylene and other moldable plastic suitable for cultivation of cells. Construction material are preferably sterilizable by irradiation, gas sterilization, autoclaving or other suitable method to assure sterility prior to use. The construction material is preferably low cost, easy to shape and sterilize, and rigid enough to prevent deformation during use or under pressure by clamping mechanisms or robotics.

In one embodiment of the present invention, the exterior length and width of the container C will preferably correspond to the ANSI/SBS (American National Standards Institute/Society for Biomolecular Screening) well plate container format; namely, the length of the container at the top is about 124 mm, the length of the container at the bottom is about 127.5 mm, the width of the container at the top is about 82 mm, and the width of the container at the bottom is about 85.5 mm. The height of the container conforms to the SBS standard for deep well plate containers or about 42 mm. As is apparent, other dimensions for the container C (e.g. square) are within the scope of the present invention so long as the container C is adapted to receive one or more cultivation vessel 10 and is capable of handling by automation.

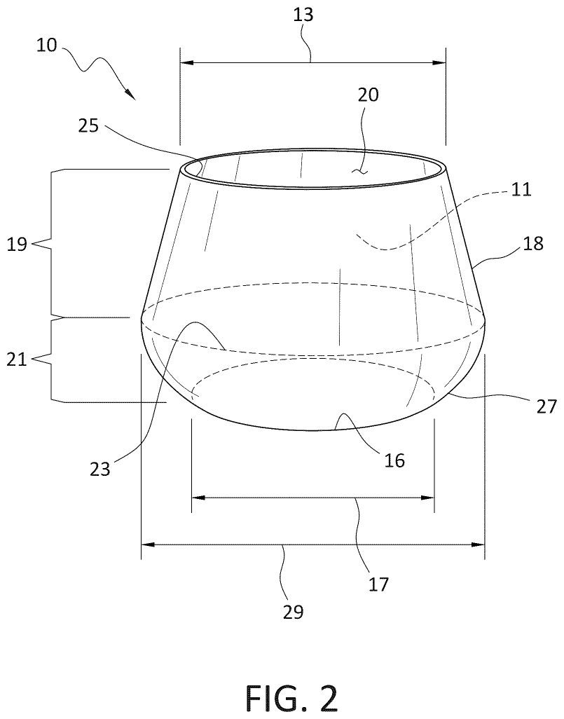

FIG. 2 shows cultivation vessel 10 in greater detail. The vessel 10 includes a bottom 16, side wall 18 and an opening 20. The side wall 18 and bottom 16 define an internal cavity 11. Opening 20 provides access to the internal cavity 11. The opening 20 has an opening diameter 13. The bottom 16 has a bottom diameter 17. The opening diameter 13 is preferred to be about equal the bottom diameter 17. It will be appreciated that in some embodiments of the invention, the opening diameter 13 and the bottom diameter 17 need not be exactly the same and one may be somewhat larger/smaller than the other.

Wall 18 is shown in FIG. 2 to include an upper portion 19 and a lower portion 21. The upper portion 19 is preferably frustoconical in shape and as generally shown in FIG. 2. The upper portion 19 of the wall joins the lower portion 21 of the wall circumferentially around the vessel 10 at join line 23. The top edge 25 of upper portion 19 has a perimeter that defines the opening 20 of the cultivation vessel 10. The bottom of upper portion 19 of the side wall is at join line 23. The top of lower portion 21 of the side wall is at join line 23 and the bottom 27 of side wall 18 lower portion 21 has a perimeter that defines the diameter 17 of bottom 16 of cultivation vessel 10. Join line 23, where the side wall upper portion 19 and lower portion 21 meet has a diameter 29. The diameter 29 of join line 23 is larger than both the diameter 13 of opening 20 and the diameter 17 of lower portions 21. The lower portion 21 transitions the side wall 18 from its larger diameter 29 to its smaller diameter 17 of bottom 16. The lower portion 21 is preferred to have a generally frustospherical shape as shown in the figures. It is within the scope of the invention to provide variations to bottom 16 including, for example, the addition of raised baffles or other structure that assist in generating turbulence to a liquid in vessel 10 during shaking.

The working volume of the cultivation vessel 10 is variable but generally greater than about 20 ml, preferably about 35 ml, and in another preferred embodiment about 125 ml. These volumes permit at least one, and up to two cultivation vessels within a single cultivation container C.

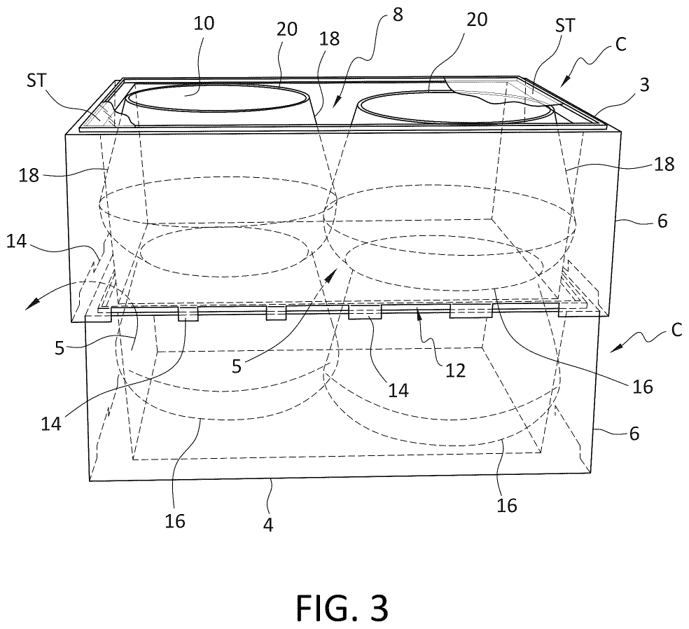

FIGS. 1 and 3 illustrate an embodiment of the invention where the container bottom 4 is provided with air/gas flow channels 12 that extend transverse to the longitudinal axis of the container C. A series of foot members 14 are provided along the perimeter of the container bottom 4 with the air/gas flow channels 12 extending between the foot members 14 The air/gas flow channels allow ambient air/gas to freely pass beneath the bottom 4 of container C and as generally shown by arrow 5. In this embodiment, the open top of container C is covered with a conventional adhesive sealing film ST that is described in more detail below. The air/gas flow channels are particularly advantageous when multiple containers C are arranged in a vertically stacked relation as will be further described below.

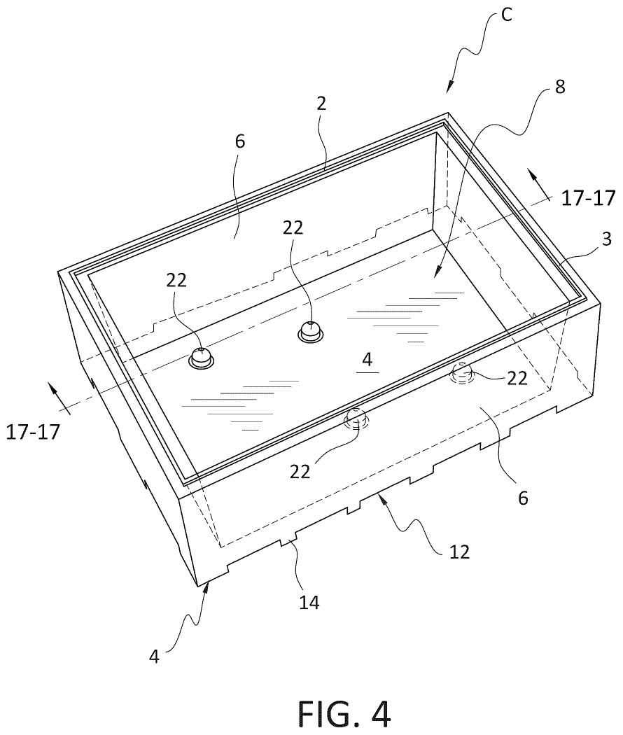

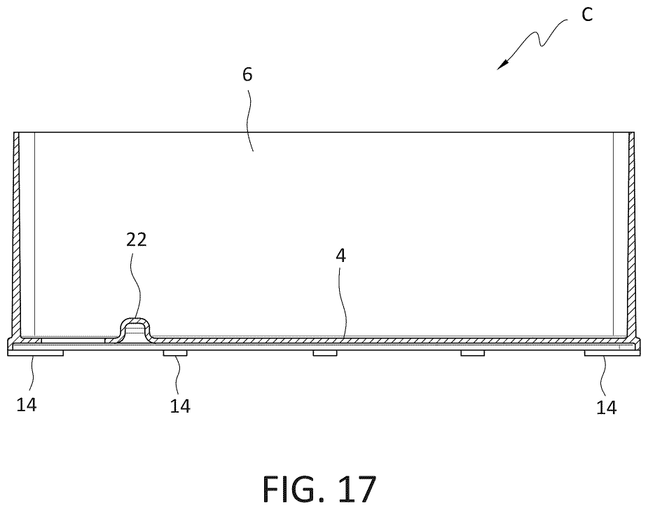

FIG. 4 shows the container C of FIG. 1 with the cultivation vessels removed so that abutment members 22 on the bottom 4 of container C can be seen. Two of the abutment members 22 secure one of the cultivation vessels 10 inside of the culture container C by wedging it or otherwise fitting it against a corner of the container C formed by intersecting side walls 6. The lower portion 21 of the cultivation vessel will contact abutment members 22 so as to effectuate the precise location of the vessel in container C. Once held in position by the abutment members 22, the vessel 10 may be fixed into place by suitable methods such as sonic welding, adhesives or by other mechanical attachment means. As best seen in FIG. 17, abutment members 22 may be molded into the bottom 4 of container C (and are therefore of the same polymer construction as the container), to precisely control the location of each vessel 10 within container C. The location of each vessel 10 within the container C is essential to ensure correct alignment with and mating to a cooperating cover member 10 as will be further explained below. As is apparent, other means and methods for securing the cultivation vessel inside the container are within the scope of the present invention so long as the cultivation vessels are securely fixed within container C and cannot shift when being shaken within the incubator.

Returning to FIG. 3, container C is shown in a vertically stacked relation with an additional container and with an adhesive sealing tape ST disposed over the top of each container. The foot members 14 that extend from the bottom of the culture container C contact the top 2 of a container C directly beneath it. The raised edge 3 projecting from the container top 2 abuts the foot members 14 of the container directly above it to prevent it from shifting laterally. The gas flow channels 12 disposed between the foot members 14 allow air/gas to be freely directed between adjacent containers as generally shown by arrow 5.

Adhesive sealing tapes/films are widely available membranes having an adhesive backing. They are typically applied across the entire top surface of a well plate. Sealing tapes are a relatively low-cost method to seal well plate vessels and prevent contamination but are also breathable i.e. they permit good gas exchange. Sealing tapes are relatively low cost; but they can be cumbersome for ongoing cultivations e.g. every time a sample needs to be withdrawn from a cultivation vessel the user must unpeel the tape which exposes the uncovered vessels to cross-contamination. The sealing tape membranes are porous yet they also maintain a sterile barrier that functions as breathable sealing film for well plates of all types. The tapes are pre-sterilized and generally single use. The user peels the backing off the tape ST and adheres it to the top 2 of the container thereby sealing its contents while allowing gas permeability into the container's vessels. Following use, the tape ST is peeled off of the container and disposed of.

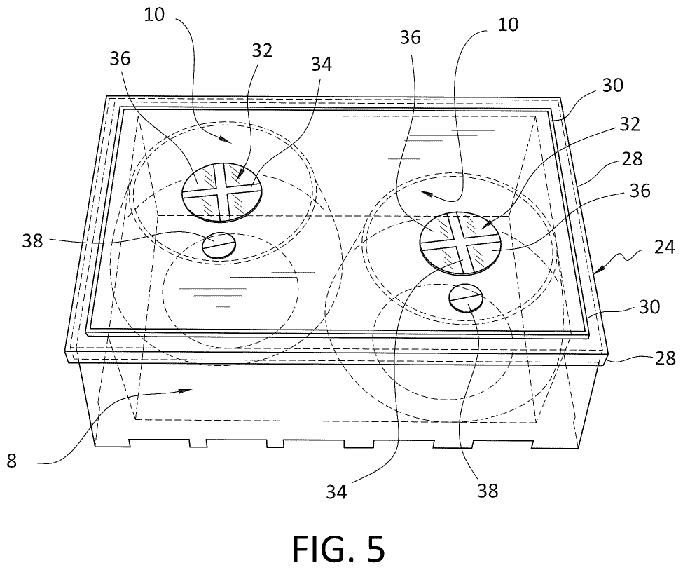

Turning to FIG. 5, the container according to the present invention is provided with a removable closure or cover member 24 secured to top 2. This embodiment eliminates the need for a sealing tape. In a preferred embodiment, the cover member 24 has a side wall 28 that extends the perimeter of the cover and overlaps an edge of the container top 2. The side wall 28 preferably has a height of about 5.0 mm to facilitate gripping by off the shelf robotics, a length of about 127.0 mm and a width of about 86.0 mm. The side wall may have a texture or rubberized coating to further facilitate robotics handling. The top surface of the cover member 24 includes a raised edge 30 shown to extend the perimeter of the cover member. Other dimensions for the cover member 24 are within the scope of the present invention so long as the cover member is adapted to tightly seal with the tops of the vessels 10 contained within container C. In a preferred embodiment, the dimensions of cover member 24 are adapted to interfit the top of a SBS well plate container.

A seal is preferably provided for both the cover and the vessels 10 located in container C. The compressible seal is preferably a mechanical type compression seal that is formed from, for example, silicone or other rubberized soft material such as thermoplastic elastomers, closed cell rubber foam, liquid silicone rubber, and die-punched silicone sheet materials. As is apparent, other mechanisms to provide an airtight seal between the cover 24 and the vessel(s) are within the scope of the invention, e.g. clamps, adhesives, etc.

A pair of vents 32 extend through the top of the cover member 24 so that when the cover is secured to a container, each vent is aligned with a separate one of the two cultivation vessels 10 in container C. The vent 32 is shown in this embodiment to include a cross-shaped member 34 that supports a sterile gas permeable membrane 36. The gas permeable membrane 36 is a sterility maintaining membrane that allows gas exchange through the membrane. By way of example, a suitable membrane 36 is 0.2 μm PVDF membrane or an ePTFE membrane. As is apparent, other membrane materials are possible provided they maintain gas transfer and a 0.2 μm barrier to prevent contaminants from entering the vessel. The membrane may also function to prevent condensation from forming on the underside of the closure and reduce dripping of condensation onto the outside of each cultivation vessel 10 (when the cover is removed) and it prevents contaminants from entering each cultivation vessel. It is within the scope of the present invention to provide other mechanisms for securing and/or supporting a gas permeable membrane over vent 32.

A pierceable septum 38 is provided to permit the addition or removal of liquid from the cultivation vessel 10 with a pipette or other device. The septum 38 is located off center from the vertical axis of the cultivation vessel. This feature, in combination with the curvature of the lower portion 21 of sidewall 18, enables a user to pipette very small amounts of liquid settling on the planar bottom 16 of the vessel 10, by tilting the container so that the liquid collects along the concave curvature of the vessels lower portion 21.

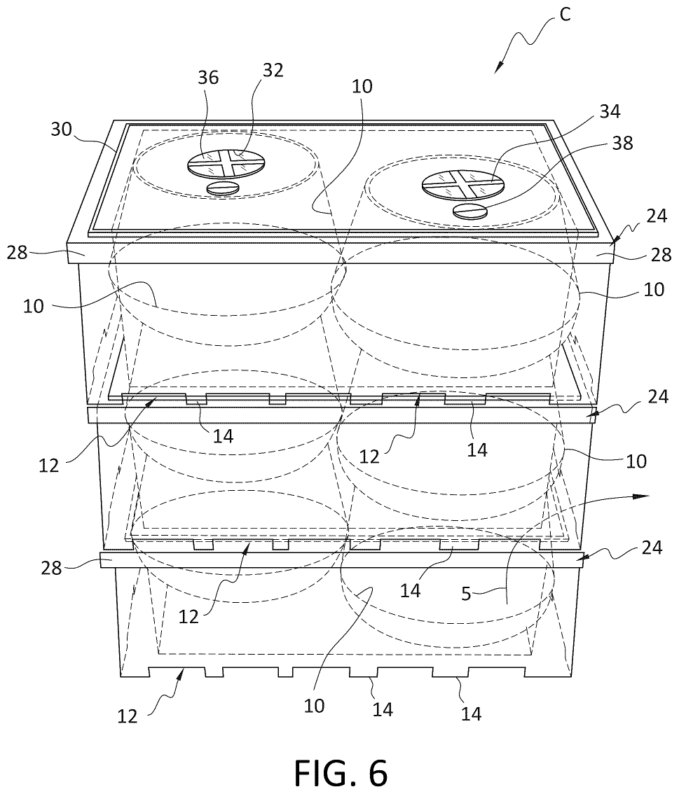

FIG. 6 illustrates three of the culture containers C shown in FIG. 5 assembled in a vertically stacked relation. The foot members 14 that extend from the bottom 4 of the culture container C contact the top surface of a cover member 24 directly beneath it. The total perimeter of the bottom of the container is slightly larger than the total perimeter of the raised edge 30 on the cover member 24 to enable two identical containers to stack on top of each other by nesting. The circumferential raised edge 30 of the cover abuts foot members 14 and prevents container C from shifting laterally. The gas flow channels 12 disposed between the foot members 14 ensure airflow to the cover of the container directly beneath and as illustrated by arrow 5.

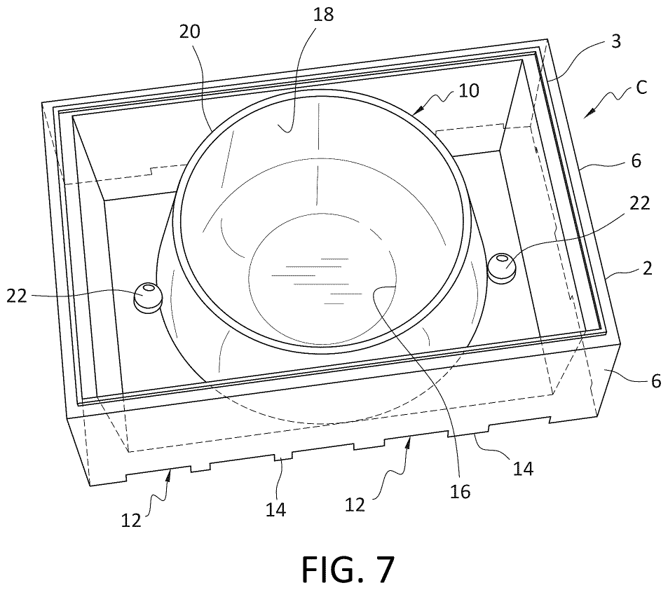

FIG. 7 illustrates another embodiment of the invention where a single cultivation vessel 10 is provided within container C of FIG. 1. In this embodiment, the cultivation vessel 10 preferably has a 250 ml working volume and otherwise corresponds to the shape of the cultivation vessel shown in FIG. 2. This embodiment may include a cover member 24 so long as the cover is adapted to engage the single cultivation vessel 10 within container C. Two (or more) abutments 22 are provided to contact the cultivation vessel 10 at its lower portion 21. Side walls 6 of the container also contact the walls 18 of the cultivation vessel 10 at two separate points to constrain the cultivation vessel at four separate contact locations and fix the vessel in place along the horizontal plane of the container.

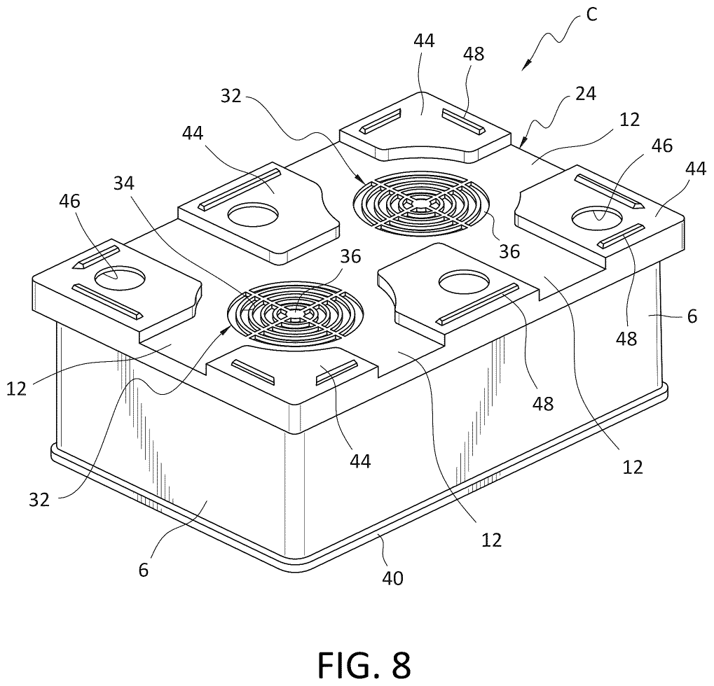

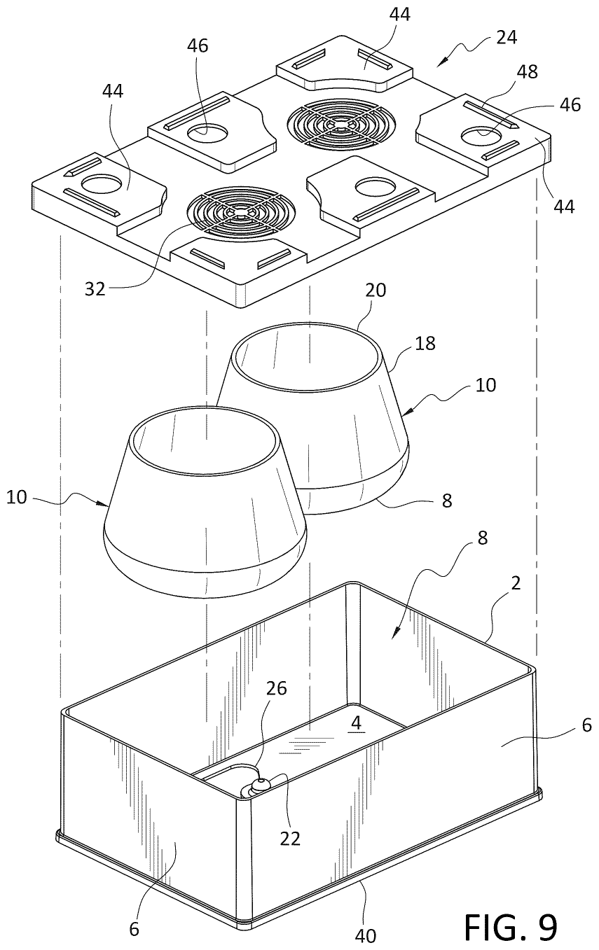

FIGS. 8 through 15 illustrate another embodiment of the invention. In this embodiment, the cover member 24 is provided with air/gas flow channels 12 so that when multiple containers C are fitted with cover members and arranged in a stacked relation, ambient air/gas will freely pass between the adjacent containers. In addition, the cover member 24 in this embodiment interfits the top edges 25 of opening 20 for each cultivation vessel 10 in container C to seal it against contamination. Also, openings 46 and 26 are provided in both the cover members 24 and the bottom of the container C to allow ambient air/gas to pass through each container and around the hermetically sealed cultivation vessels within the container.

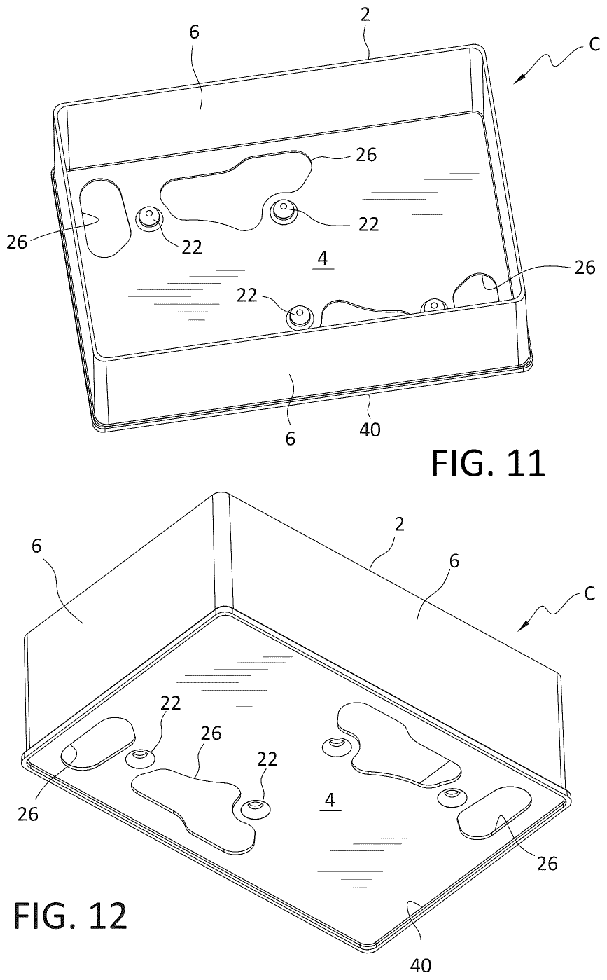

FIGS. 11 and 12 show container C is provided with openings 26 disposed in bottom surface 4 to allow air/gas to enter the container's interior. The openings 26 may vary in size and/or number from that as shown in the figures. Preferably, the openings 26 are disposed in regions of bottom surface 4 other than where the cultivation vessels 10 are located. Other shaped openings or passageways are within the scope of the present invention so long as the transmission of air/gas flow through the container is deemed sufficient. The perimeter of the container bottom 4 is provided with a flange member 40 that extends from the container side walls 6 and is adapted to interfit the abutments 48 of cover member 24 of a separate container as will be further explained below.

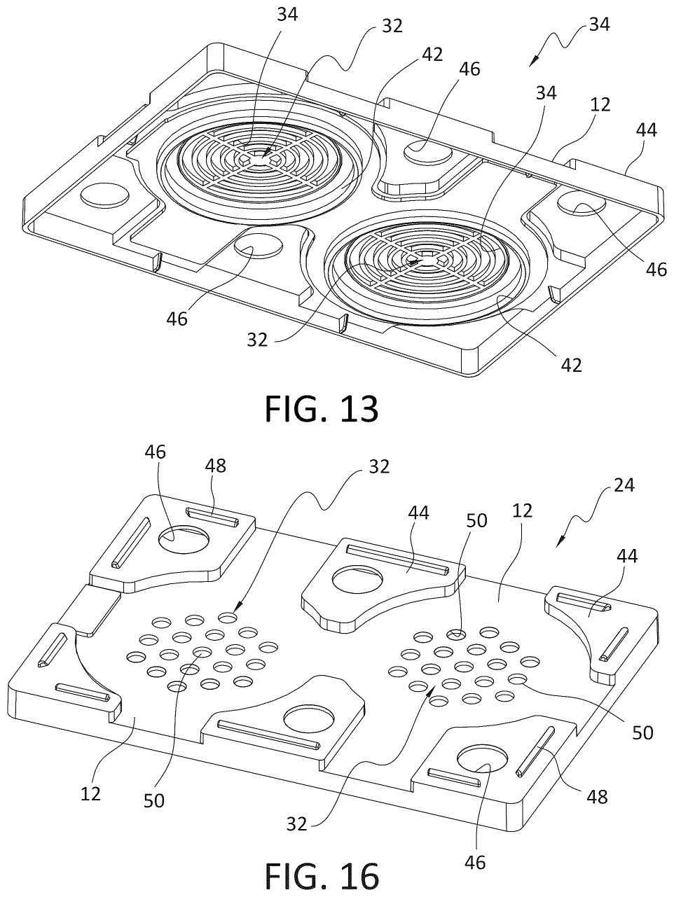

FIGS. 8 and 13 illustrate cover member 24 in greater detail. The cover member 24 is provided with a pair of vents 32 extending through the cover member 24 so that when the cover is secured to container C, each vent 32 is aligned with a separate one of cultivation vessels 10 inside the container C. The vents 32 in this embodiment are seen to comprise a series of spaced apart concentric ring members 34 that support a gas permeable membrane 36. The gas permeable membrane 36 enables gas to exit each cultivation vessel but prevents contaminants from entering it as described earlier. The gas permeable membrane may be fixed to the cover by a variety of methods including, for example, sonic welding, heat staking or adhesives. The membrane may also be held firmly in place over the vent by a retaining ring that is press fit into a cooperating retainer incorporated into the cover. The main objective when fixing the membrane is to ensure it has adequate support and is protected from damage in the case of inadvertent contact. It is within the scope of the present invention to provide other vents 32 from that as shown. For example, FIG. 16 illustrates an alternative embodiment where the vent 32 comprises a series of apertures 50. Venting in this embodiment may be increased or decreased by modifying the surface area of the openings that comprise the vent.

FIG. 13 illustrates the underside of cover 24 may be provided with gaskets 42 that are coaxially aligned with each of the vents 32. The gasket interfits the mouth 20 at top edge 25 of the cultivation vessels 10 to provide an airtight seal. The gaskets 42 may be a separate member that is press fit into a receiving groove or unitary with the cover as shown in FIG. 13. The gaskets may be compression fit into a receiving groove provided on the underside of the cover, or it may be secured by over molding directly to the lid or it may be secured with an adhesive. The construction material of the gasket are as described earlier; namely, silicone or some other rubberized soft material such as thermoplastic elastomers, closed cell rubber foam, liquid silicone rubber, and die-punched silicone sheet materials.

A gas permeable membrane 36 is associated with each vent 32. The gas permeable membrane enables gas to enter and to exit the cultivation vessel but prevents contaminants from entering. The gas permeable membrane is a sterility maintaining membrane that allows gas exchange through the membrane. For example, a suitable membrane 36 is 0.2 μm PVDF membrane or an ePTFE membrane. The membrane also serves to prevent any condensation from forming on the underside of the closure and reduce dripping onto the outside of each cultivation vessel 10, it also prevents contaminants from entering the cultivation vessel.

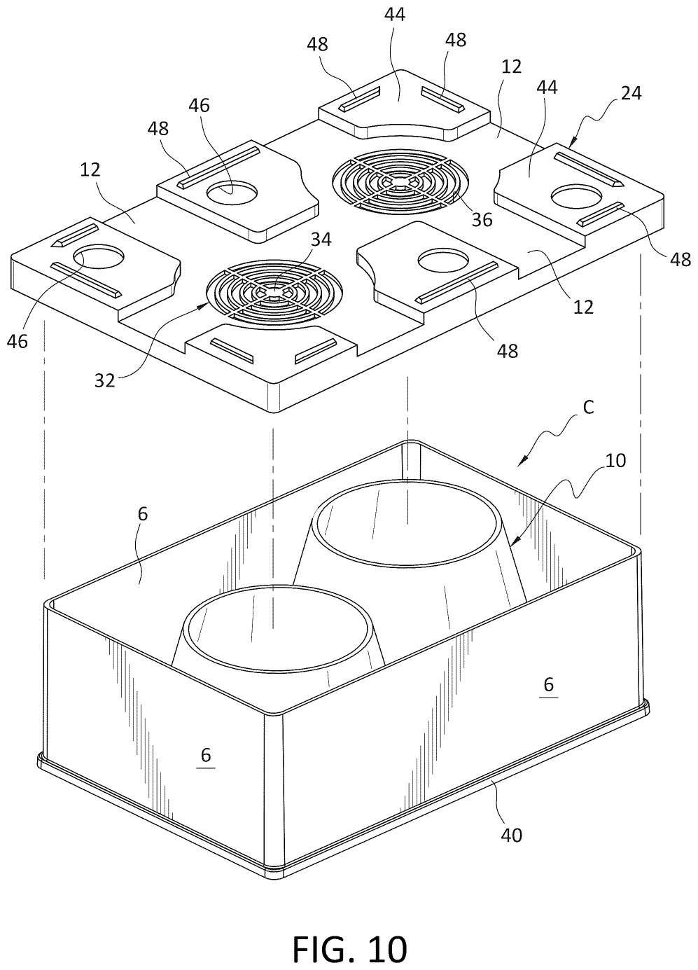

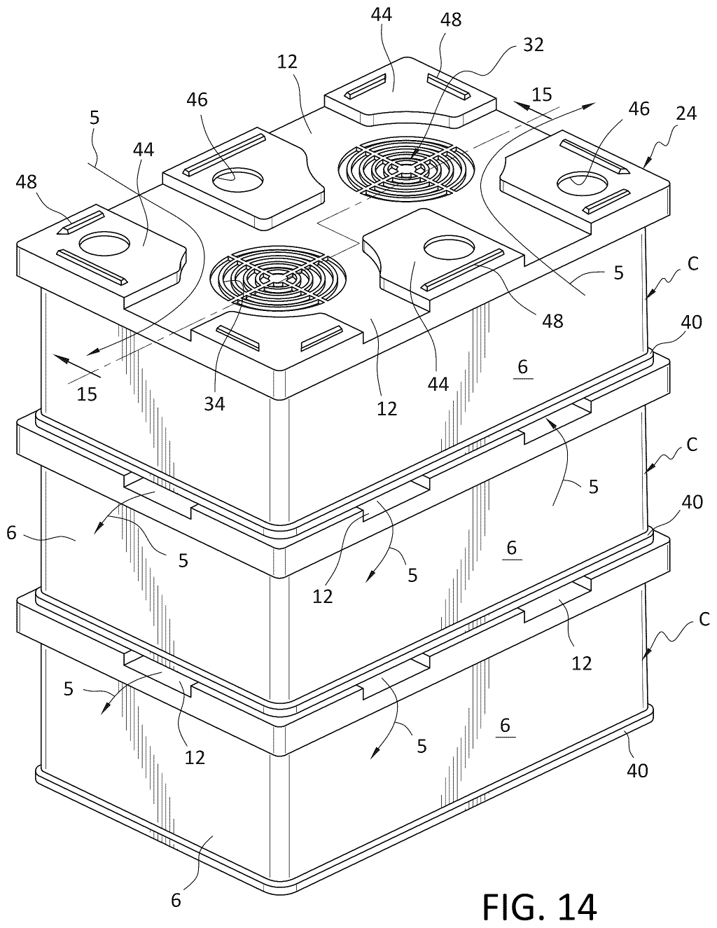

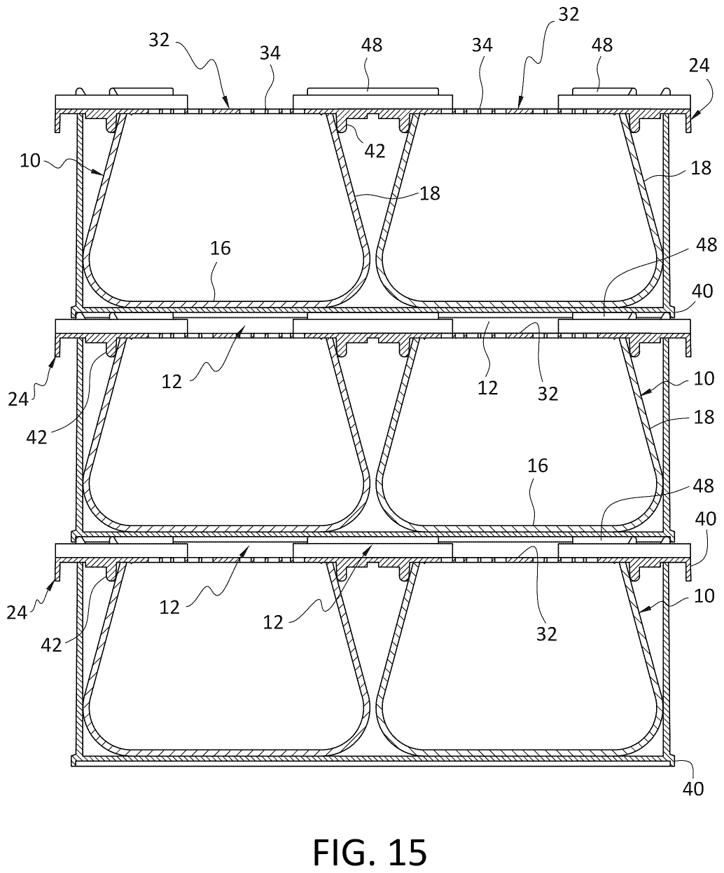

As best shown in FIGS. 8 and 10, a series of openings 46 extend through cover member 24. The cover member openings 46 and the container openings 26 in the bottom 4 of the container cooperate to enable air/gas to freely enter and exit the interior of each container. The top surface of cover 24 is also provided with several air/gas flow channels 12 that extend between raised portions 44. When multiple containers C are aligned in a vertically stacked relation (FIGS. 14 and 15), air/gas may pass between the bottom 4 of a container and the lid 24 of a container directly beneath it. The air/gas pathway through the various channels 12 is best shown by arrow 5 in FIG. 14. Other openings or passageways are within the scope of the present invention so long as the transmission of air/gas flow through the container is deemed sufficient. For example, an opening may be provided within the vertical side walls 6 of the container shown in FIG. 10 to further promote airflow in and around the cultivation vessels when multiple containers are arranged in a vertically stacked alignment or when otherwise adjacent to one another in the incubator.

The raised portions 44 on the cover member 24 include abutments 48. As best shown in FIG. 14, the abutments 48 prevent the containers C from shifting laterally when in a stacked alignment by engaging against flange members 40 that extend from the bottom of each container.

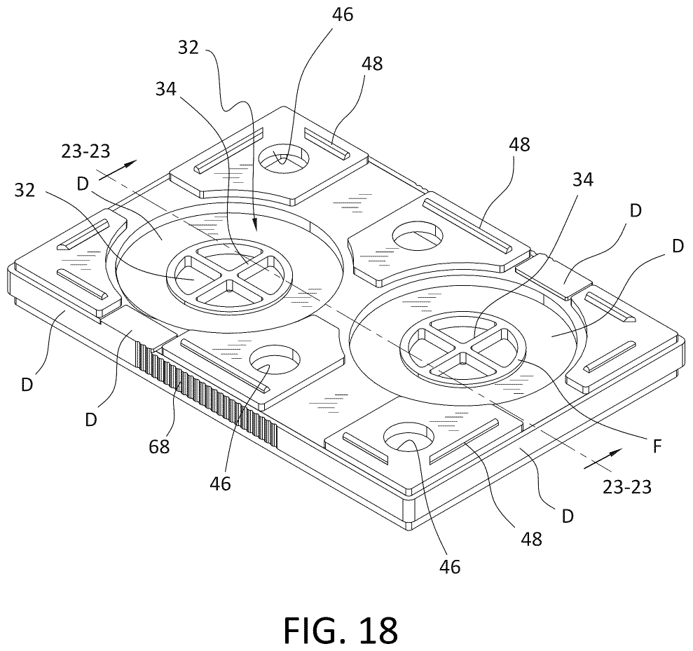

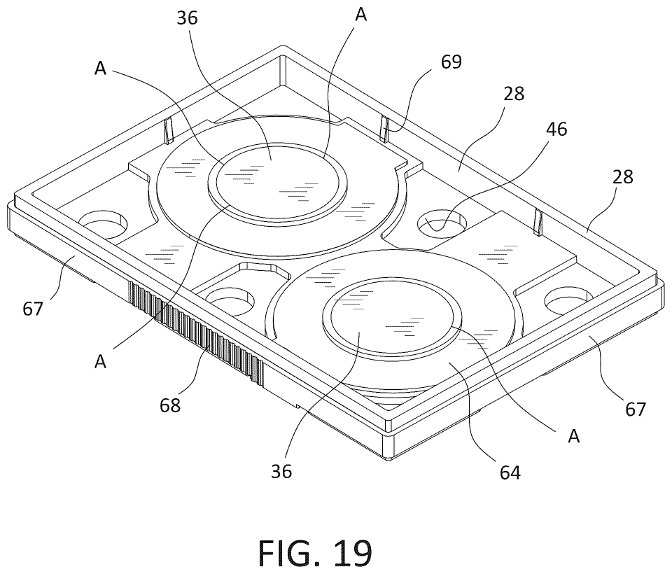

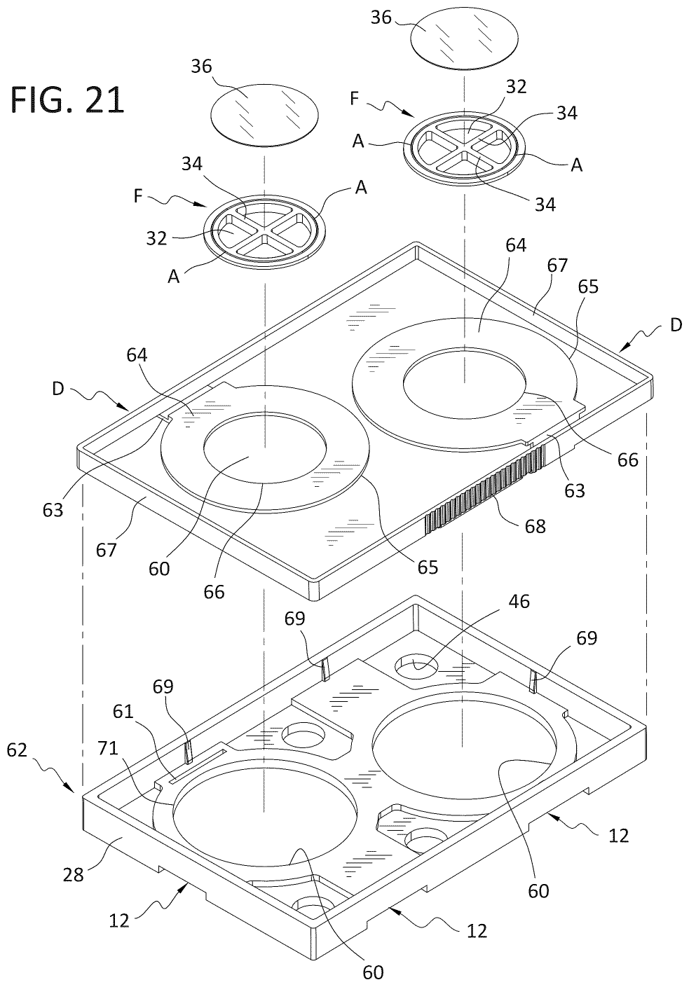

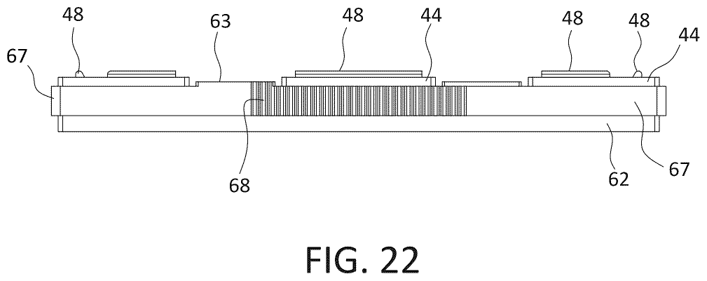

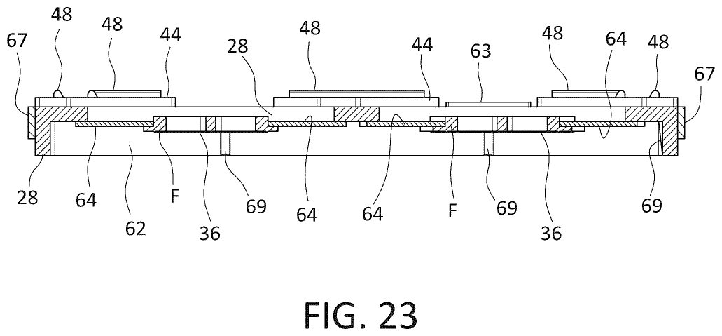

FIGS. 18 through 23 illustrate another embodiment of the cover member 24 according to the present invention. The cover member 24 in this embodiment is composed of a frame 62, a gasket overmold D including outer rim 67, a pair of islands F, and gas permeable membranes 36 overlying the support members 34 of each vent 32. The frame 62 is provided with sidewalls 28 that will preferably have a height of at least about 5 mm. The gasket overmold D including its outer rim 67 are overmolded onto the sidewalls 28 of cover frame 62. The gasket overmold D is preferably composed of silicon, a thermoplastic elastomer or other material having rubber-like characteristics. Location members 69 are provided along the interior perimeter walls 28 of frame 62 so that the cover member 24 can be precisely positioned onto a container C and sealingly engage the cultivation vessels disposed within the container.

Slots 61 are provided in the cover frame 62 to secure the gasket overmold D to frame 62. Tab 63 of the gasket overmold D extends through each respective slot 61 to secure the gasket overmold D into place and to attach the outer rim 67 of the gasket overmold D to gasket area 64. The outer rim 67 may include a textured surface 68 that enhances the gripping characteristics of the cover when handled by a robotic system. Gasket area 64 of the gasket overmold D includes an inner edge 66 and an outer edge 65 that respectively engage edge feature 70 of island F and edge feature 71 of the cover frame 62.

Each gas permeable membrane 36 may be secured to a separate one of the islands F by heat staking or other methods as described earlier. Heat Staking is done by heating an aluminum piece that is pressed into the perimeter of the membrane 36 while it is positioned against the underside of island F. The heated aluminum piece melts the plastic and fuse the membrane 36 in place on the island F. An extruded energy director ring A is provided on the bottom surface of island F to enable the plastic to melt in a controlled and reliable manner. The energy director ring A will also allow the membrane 36 to be secured to the island F by sonic welding. Once attached, the gas permeable membrane 36 with islands F in combination with gasket area 64 provide a hermetic seal to cultivation vessels 10 disposed inside the container C and also prevent sample contamination while allowing gas to exit the cultivation vessel. In addition, varying the thickness and materials of gasket 64 of the gasket overmold D gives the advantage of reducing the forces required to create a seal between the cover 24 and the cultivation vessels 10.

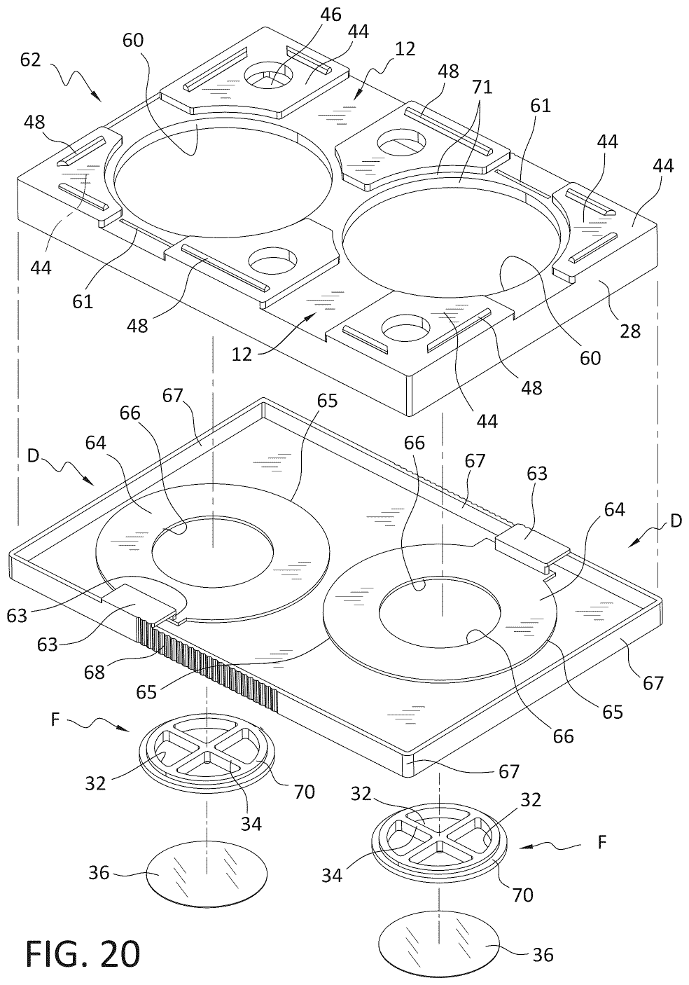

As best shown in FIG. 20, the top surface of cover member 24 includes raised portions 44 that provide a series of air/gas flow channels 12 when a separate container C is stacked onto the cover member 24, and abutments 48 that prevent lateral shifting of the containers when in a vertically stacked relation are as described above with respect to other embodiments. Also, the openings 46 in cover member 24 allow air/gas flow through the cavity of a container to which the cover member is secured and as described earlier with respect to other embodiments.

While this invention has been described as having a preferred design, it is understood that it is capable of further modifications, uses and adaptations, both in whole and in part, while following the general principle of the invention including such departures from the present disclosure as is known or customary practice in the art to which this invention pertains, and as may be applied to the central features of this invention.

What is claimed is:

1. A culture container comprising:
a) a top, a bottom and a sidewall that define an interior region, the top having an opening to the interior region; and
d) at least one cultivation vessel, the at least one cultivation vessel having an open top, a bottom and a sidewall that define an interior region, each of the open top, bottom and sidewall have a diameter, the diameters of the bottom and open top are less than the diameter of the sidewall, the sidewall having a generally frustoconical shape.

2. The culture container as in claim 1 and wherein the sidewall of the at least one cultivation vessel is in contact with the container sidewall.

3. The culture container as in claim 1 and wherein the diameters of the at least one cultivation vessel bottom and top are about the same.

4. The culture container as in claim 1 and wherein the open top of the at least one cultivation vessel is adjacent the opening of the culture container.

5. The culture container as in claim 1 further comprising no more than one additional cultivation vessel, each of the at least one cultivation vessel and the one additional cultivation vessel are in contact with the container sidewall at two separate locations.

6. The culture container as in claim 1 and further comprising foot members, the foot members extend around a perimeter of the container bottom, the foot members are adapted to interfit the top of a separate culture container.

7. The culture container as in claim 1 and further comprising at least one opening through the container bottom.

8. The culture container as in claim 1 and further comprising at least one abutment, the at least one abutment is disposed on the container bottom and is in contact with the at least one cultivation vessel.

9. The culture container as in claim 1 and further comprising a removable cover member, the cover member having a top surface and a bottom surface, the bottom surface is adapted to close the opening of the container and sealingly engage with the open top of the at least one cultivation vessel, the top surface is adapted to engage with the bottom of a separate culture container.

10. The culture container as in claim 9 and further comprising at least one vent with a gas permeable membrane, the at least one vent with gas permeable membrane extends through the cover member and is adapted to sealingly engage the at least one cultivation vessel.

11. The culture container as is claim 9 and further comprising openings, the openings extend through the cover member to allow gas to pass through the container.

12. The culture container as in claim 9 and further comprising channels, the channels are disposed on a top surface of the cover member so that when the cover is engaged with the bottom of a separate culture container gases may pass through the cover member channels.

13. The culture container as in claim 12 and further comprising abutments, the abutments are provided on the top surface of the cover member and are adapted to engage the bottom of a separate culture container.

14. The culture container as in claim 1 and wherein the container and the at least one cultivation vessel are constructed from a moldable plastic selected from the group consisting of polycarbonate, polystyrene, polyethylene terephthalate glycol and polypropylene.

15. The culture container as in claim 1 and wherein the dimensions of the container correspond to SBS well plate container format.

16. A culture container comprising:
a) a top, a bottom and four sidewalls that define an interior region, the top having an opening to the interior region and the container has a rectangular shape; and
d) a pair of cultivation vessels, each of the pair of cultivation vessel has an open top, a bottom and a sidewall that define an interior region, each of the pair of cultivation vessels open tops, bottoms, and sidewalls have a diameter, the diameters of the bottoms and open tops are less than the diameters of the sidewalls, the sidewalls have a generally frustoconical shape, and each cultivation vessel is in contact with a separate pair of the four sidewalls.

17. The culture container as in claim 16 and wherein each of the two cultivation vessels has a working volume from about 35 ml to about 125 ml.

18. The culture container as in claim 16 and wherein the dimensions of the container correspond to SBS well plate container format.

19. A culture container comprising:
a) a top, a bottom and a sidewall that define an interior region, the top having an opening to the interior region; and
d) at least one cultivation vessel, the at least one cultivation vessel having an open top, a bottom and a sidewall that define an interior region, the sidewall having an upper portion of a generally frustoconical shape and a lower portion of a generally frustospherical shape, each of the open top, bottom and sidewall have a diameter, the diameters of the bottom and open top are less than the diameter of the sidewall where the upper and lower portion meet.

20. The culture container as in claim 19 and wherein the dimensions of the container correspond to SBS well plate container format.
